(19) United States
(12) Patent Application Publication
(10) Pub. No.: US 2020/0106571 A1
(43) Pub. Date: Apr. 2, 2020

(54) DATA COMMUNICATION SYSTEM AND DATA COMMUNICATION APPARATUS

(71) Applicants: OMRON HEALTHCARE CO., LTD., Muko-shi (JP); OMRON Corporation, Kyoto-shi (JP)

(72) Inventors: Nobuo KUBO, Kyoto (JP); Toru DENO, Kyoto (JP); Hideki KONDO, Kyoto (JP)

(21) Appl. No.: 16/702,715

(22) Filed: Dec. 4, 2019

Related U.S. Application Data
(63) Continuation of application No. PCT/JP2018/028816, filed on Aug. 1, 2018.

(30) Foreign Application Priority Data
Aug. 9, 2017 (JP) 2017-154756

(51) Int. Cl. H04L 1/22; H04W 88/04; A61B 5/00

(52) U.S. Cl. CPC H04L 1/22; A61B 5/002; H04W 88/04

(57) ABSTRACT

According to an aspect of the invention, a data communication system includes first and second data communication apparatuses, and a data server. The first data communication apparatus receives first data transmitted from a data transmitting apparatus through a unidirectional communication, receives second data transmitted from the data server through a bidirectional communication, and generates output data based on at least one of the first and second data. The second data communication apparatus receives the second data transmitted from the data transmitting apparatus through a unidirectional communication, and transmits the second data to the data server through a bidirectional communication.

This application is a Continuation Application of PCT Application No. PCT/JP2018/028816, filed Aug. 1, 2018 and based upon and claiming the benefit of priority from Japanese Patent Application No. 2017-154756, filed Aug. 9, 2017, the entire contents of all of which are incorporated herein by reference.

### FIELD

The present invention relates to a data communication system and a data communication apparatus.

### BACKGROUND

Blood pressure monitors equipped with a function of transferring blood pressure data to a user's portable information terminal have been launched onto the market. As the portable information terminal, a smartphone, a tablet terminal, or a notebook personal computer, for example, is used. By employing such a function, the user can view, on the portable information terminal, the results of the measurements of the amounts related to the user's blood pressures in various situations. For the transfer of blood pressure data, short-range wireless communication technology is typically used, Bluetooth (registered trademark) in particular. In general, Bluetooth-based communications (connections) can be implemented on a small scale and in a manner which saves power, as compared to wireless local area network (WLAN) communications. The Bluetooth specification Version 4.0, also called Bluetooth Low Energy (BLE), is capable of further reducing the power consumption, as compared to the legacy specifications.

BLE allows for bidirectional communications called “connections”. However, connections come with certain problems, such as the complexity of operations required of users for pairing; the complexity of communication procedures following the pairing; the necessity for the portable information terminal side to support BLE; the necessity for the blood pressure monitor, as well as the portable information terminal, to install high-performance hardware (processor, memory, etc.); high development and appraisal costs; and unsuitability for low-capacity data transmissions due to the heaviness of the communication overhead.

On the other hand, BLE also allows for unidirectional communications called “advertising”. Japanese Patent No. 5852620 discloses a technique of transmitting an advertisement packet by including given data in a margin area of its data field.

### SUMMARY

If the blood pressure monitor transmits blood pressure data through the employment of advertising, a portable information terminal capable of receiving advertisements can receive the blood pressure data without the need for pairing or subsequent complicated communication procedures.

However, in such unidirectional communications, there is a risk that the portable information terminal may fail to receive the blood pressure data, depending on the communication situation. For example, if the blood pressure monitor is equipped only with a unidirectional transmission function, the state of the portable information terminal (e.g., the data reception status) cannot be referred to from the blood pressure monitor. Thus, there is a risk that the portable information terminal may fail to receive the blood pressure data.

An object of the present invention is to provide a technique for increasing the opportunity to receive data transmitted through unidirectional communication, thus compensating for reception failures.

According to a first aspect of the invention, a data communication system comprises first and second data communication apparatuses, and a data server which communicates with the first and second data communication apparatuses, wherein the first data communication apparatus receives first data transmitted from a data transmitting apparatus through a unidirectional communication, receives second data transmitted from the data server through a bidirectional communication, and generates output data based on at least one of the first and second data, the second data communication apparatus receives the second data transmitted from the data transmitting apparatus through a unidirectional communication, and transmits the second data to the data server through a bidirectional communication, and the data server receives the second data transmitted from the second data communication apparatus through a bidirectional communication, and transmits the second data to the first data communication apparatus through a bidirectional communication.

According to the data communication system of the first aspect, since the first data communication apparatus is provided with an opportunity to receive data from the data transmitting apparatus either directly or indirectly through the employment of both unidirectional and bidirectional communications, it is possible to increase the opportunity to receive data from the data transmitting apparatus. In the first data communication apparatus, since the output data is generated based on at least one of the first data received through a unidirectional communication with the data transmitting apparatus and the second data (transmitted by the data transmitting apparatus) received through a bidirectional communication with the data server, it is possible to compensate for some or all of the data reception failures (or increase the possibility of the compensation) when reception of at least one of the first data and the second data has failed. When at least one of the first data and the second data cannot be received by the first data communication apparatus due to the communication status, the output data can be generated based on the other data that has been received.

According to a second aspect of the invention, the first data communication apparatus generates the output data by removal of one of the duplicated items of data included in the first and second data.

According to the data communication system of the second aspect, since output data is created in the first data communication apparatus by the removal, if applicable, of one of the duplicated items of data included in the output data, it is possible to overcome the inconveniences caused by the inclusion, such as the increase in the amount of output data and the difficulty of utilizing the output data.

According to a third aspect of the invention, the first and second data include biological data.

According to the data communication system of the third aspect, it is possible, when reception of biological data has failed, to compensate for some or all of the reception failures of the biological data (or increase the possibility of the compensation) in the first data communication apparatus.

According to a fourth aspect of the invention, the first data communication apparatus receives the second data from the data server through a periodic bidirectional communication.

According to the data communication apparatus of the fourth aspect, it is possible to increase the opportunity for the first data communication apparatus to receive second data through a periodic bidirectional communication, thereby increasing the possibility of receiving the second data.

According to a fifth aspect of the invention, the first data communication apparatus transmits the first data to the data server through the bidirectional communication, based on the reception of the first data.

According to the data communication apparatus of the fifth aspect, it is possible to allow the first data communication apparatus to relay first data, transmitted from the data transmitting apparatus through a unidirectional communication, to a data server through a bidirectional communication. This allows the data server, which cannot directly receive the first data transmitted from the data transmitting apparatus through a unidirectional communication, to receive the first data transmitted from the data transmitting apparatus.

According to a sixth aspect of the invention, a data communication apparatus comprises a receiving unit which receives first data transmitted from a data transmitting apparatus through a unidirectional communication, a transmitting and receiving unit which receives second data transmitted from a data server through a bidirectional communication, a data generating unit which generates output data based on at least one of the first and second data, the second data is data transmitted from the data transmitting apparatus through the unidirectional communication, received by another data communication apparatus different from the data communication apparatus, and transmitted to the data server from said another data communication apparatus.

According to the data communication apparatus of the sixth aspect, since an opportunity is provided to receive data from the data transmitting apparatus either directly or indirectly through the employment of both unidirectional and bidirectional communications, it is possible to increase the opportunity to receive data from the data transmitting apparatus. Since the output data is generated based on at least one of the first data received through a unidirectional communication with the data transmitting apparatus and the second data (transmitted by the data transmitting apparatus) received through a bidirectional communication with the data server, it is possible to compensate for some or all of the data reception failures (or increase the possibility of the compensation) when reception of at least one of the first data and the second data has failed. When at least one of the first data and the second data cannot be received due to the communication status, the output data can be generated based on the other data that has been received.

According to a seventh aspect of the invention, the data generating unit generates the output data by removal of one of duplicated items of data included in the first and second data.

According to the data communication apparatus of the seventh aspect, since output data is created by the removal, if applicable, of one of the duplicated items of data included in the output data, it is possible to overcome the inconveniences caused by the inclusion, such as the increase in the amount of, and difficulty of utilizing the output data.

According to an eighth aspect of the invention, the first and second data include biological data.

According to the data communication apparatus of the eighth aspect, it is possible, when reception of biological data has failed, to compensate for some or all of the reception failures of the biological data (or to increase the possibility of the compensation).

According to a ninth aspect of the invention, the transmitting and receiving unit receives the second data from the data server through a periodic bidirectional communication.

According to the data communication apparatus of the ninth aspect, it is possible to increase the opportunity to receive second data through a periodic bidirectional communication, thereby increasing the possibility of receiving the second data.

According to a tenth aspect of the invention, the data communication apparatus comprises a relay control unit which performs control to transmit the first data to the data server through the bidirectional communication, based on the reception of the first data by the receiving unit.

According to the data communication apparatus of the tenth aspect, it is possible to relay first data, transmitted from the data transmitting apparatus through a unidirectional communication, to a data server through a bidirectional communication. This allows the data server, which cannot directly receive the first data transmitted from the data transmitting apparatus through a unidirectional communication, to receive the first data transmitted from the data transmitting apparatus.

According to the present invention, it is possible to provide a technique for increasing the opportunity to receive data transmitted through a unidirectional communication, thus compensating for reception failures.

### BRIEF DESCRIPTION OF THE DRAWINGS

FIG. 1 is a diagram schematically showing an application example of a data communication system according to the present embodiment.

FIG. 2 is a conceptual diagram showing an example of the data communication system according to the present embodiment.

FIG. 3 is a block diagram showing an example of a hardware configuration of a data transmitting apparatus according to the present embodiment.

FIG. 4 is a block diagram showing an example of a software configuration of the data transmitting apparatus according to the present embodiment.

FIG. 5 is a block diagram showing an example of a hardware configuration of the data communication apparatus according to the present embodiment.

FIG. 6 is a block diagram showing an example of a software configuration of the data communication apparatus according to the present embodiment.

FIG. 7 is a block diagram showing an example of a hardware configuration of a data server according to the present embodiment.

FIG. 8 is a block diagram showing an example of a software configuration of the data server according to the present embodiment.

FIG. 9 is a diagram illustrating advertising performed in BLE.

FIG. 10 is a diagram illustrating a data structure of a packet transmitted and received in BLE.

FIG. 11 is a diagram illustrating a data structure of a PDU field of an advertisement packet.

FIG. 12 is a flowchart showing an example of a flowchart illustrating an example of a data reception operation of the data communication apparatus according to the embodiment.

FIG. 13 is a flowchart showing an example of a relay operation of a data relay device according to the embodiment.

### DETAILED DESCRIPTION

Hereinafter, an embodiment according to an aspect of the present invention (also referred to as “present embodiment” hereinafter) will be described, with reference to the drawings.

Elements that are the same as, or similar to, elements already described will be denoted by the same or similar reference numerals, and redundant descriptions will be basically omitted.

### § 1 APPLICATION EXAMPLE

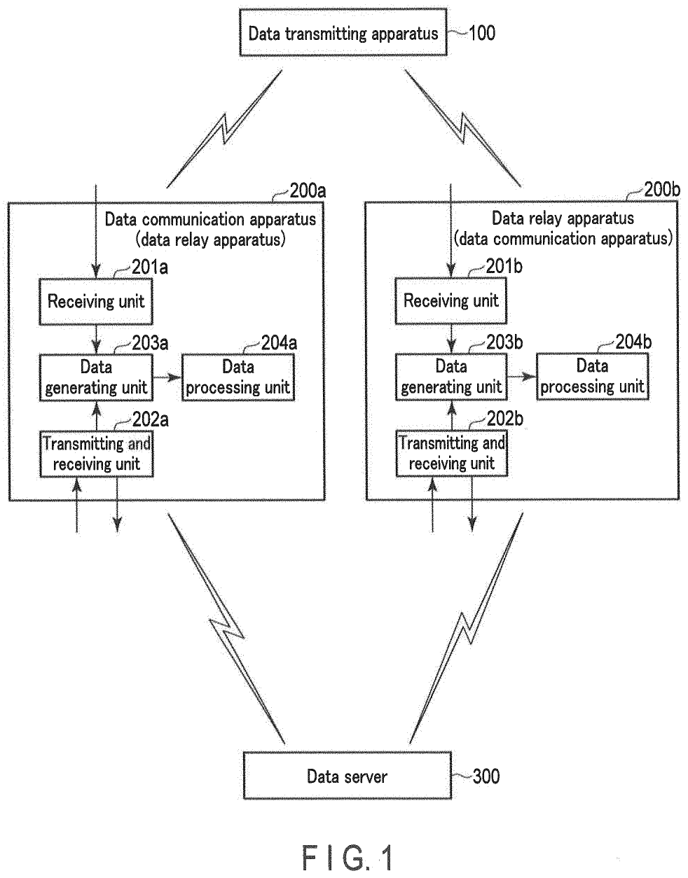

An application example of the present invention will be described, with reference to FIG. 1. FIG. 1 schematically shows an application example of a data communication system according to the present embodiment. As shown in FIG. 1, the data communication system includes a data transmitting apparatus 100, a data communication apparatus 200a, a data relay apparatus 200b, and a data server 300. The data communication apparatus (first data communication apparatus) 200a may operate as a data relay apparatus, and the data relay apparatus 200b may operate as a data communication apparatus (second data communication apparatus). For example, the data communication apparatus 200a and the data relay apparatus 200b may have the same configuration and respectively operate as a data relay apparatus and a data communication apparatus.

The data communication apparatus 200a includes at least a receiving unit 201a, a transmitting and receiving unit 202a, a data generating unit 203a, and a data processing unit 204a. The data relay apparatus 200b includes at least a receiving unit 201b, a transmitting and receiving unit 202b, a data generating unit 203b, and a data processing unit 204.

The units of the data communication apparatus 200a (the receiving unit 201a, the transmitting and receiving unit 202a, the data generating unit 203a, and the data processing unit 204a) correspond to, for example, the respective units of the data relay apparatus 200b (the receiving unit 201b, the transmitting and receiving unit 202b, the data generating unit 203b, and the data processing unit 204b); thus, the description of the operation of each unit of the data relay apparatus 200b will be omitted.

The receiving unit 201a receives first data transmitted from the data transmitting apparatus 100. The data transmitting apparatus 100 transmits, through a unidirectional communication compliant with communication standards such as BLE, a radio signal that carries the packet defined by the standard, and the receiving unit 201a receives the radio signal. The receiving unit 201a sends the received signal to the data generating unit 203a. Since the data transmitting apparatus 100 constantly transmits, for example, a radio signal that carries the packet through a unidirectional communication, when the data communication apparatus 200a enters a communication area of the data transmitting apparatus 100, the receiving unit 201a of the data communication apparatus 200a receives the radio signal.

The transmitting and receiving unit 202a receives the second data transmitted from the data server 300. The data server 300 transmits the second data through a bidirectional communication compliant with communication standards such as mobile communications (3G, 4G, etc.) and WLAN, and the transmitting and receiving unit 202a receives the second data. The transmitting and receiving unit 202a sends the received signal to the data generating unit 203a. For example, the transmitting and receiving unit 202a attempts an access to the data server 300 through a periodic bidirectional communication, and receives the second data transmitted from the data server 300.

The data generating unit 203a generates output data based on at least one of the first data and the second data. The data generating unit 203a generates the output data by, for example, removing one of the duplicated items of data included in the first and second data. The data generating unit 203a sends the output data to the data processing unit 204a.

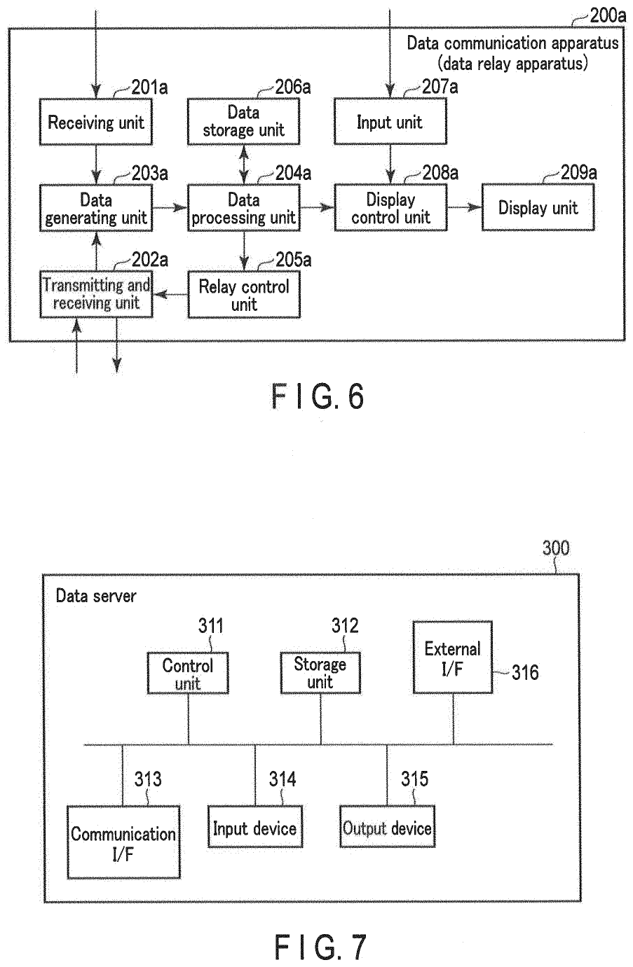

The data processing unit 204a sends the output data to a data storage unit (a data storage unit 206a, to be described later with reference to FIG. 6) and a display control unit (a display control unit 208a, to be described later with reference to FIG. 6). The data storage unit 206a stores the output data. The display control unit 208a generates display data based on the output data.

The second data is data transmitted from the data transmitting apparatus 100 through a unidirectional communication, received by the data relay apparatus 200b (corresponding to another data communication apparatus different from the data communication apparatus 200a), and transmitted to the data server 300 by the data relay apparatus 200b for transmission to a transmission destination of the data communication apparatus 200a designated in advance. That is, the data server 300 receives the second data transmitted from the data relay apparatus 200b, and transmits the received second data to the transmission destination of the data communication apparatus 200a designated in advance.

Both the first and second data may include, for example, biological data, which may include blood pressure data.

The data communication apparatus 200a receives the first data transmitted from the data transmitting apparatus 100 through a unidirectional communication, which does not require pairing or complicated communication procedures. The data communication apparatus 200a receives either a subset or the entirety the first data. The subset or entirety of the first data received by the data communication apparatus 200a will also be referred to as “first reception data”.

The data relay apparatus 200b also receives the first data transmitted from the data transmitting apparatus 100 through a unidirectional communication, which does not require pairing or complicated communication procedures. The data relay apparatus 200b receives a subset or the entirety of the first data. The subset or entirety of the first data received by the data relay apparatus 200b will also be referred to as “second reception data”. The data server 300 receives the second reception data transmitted from the data relay apparatus 200b, and transmits the second reception data to the data communication apparatus 200a, through a bidirectional communication. The data communication apparatus 200a receives the second reception data through a bidirectional communication.

Thus, the data communication apparatus 200a receives the first reception data through a unidirectional communication, and receives the second reception data through a bidirectional communication. As described above, the first reception data is a subset or the entirety of the first data transmitted from the data transmitting apparatus 100, and becomes the subset or entirety of the first data according to the state of communications between the data transmitting apparatus 100 and the data communication apparatus 200a. For example, if the state of communications is favorable (and unlikely to cause loss of data), the first reception data is highly likely to be the entirety of the first data; if the state of communications is not favorable (and likely to cause loss of data), the first reception data is highly likely to be a subset of the first data.

The second reception data is a subset or the entirety of the first data transmitted from the data transmitting apparatus 100, and becomes the subset or entirety of the first data according to the state of communications between the data transmitting apparatus 100 and the data relay apparatus 200b. For example, if the state of communications is favorable (and unlikely to cause loss of data), the second reception data is highly likely to be the entirety of the first data, and if the state of communications is not favorable (and likely to cause loss of data), the second reception data is highly likely to be a subset of the first data. Even if the first reception data is a subset of the first data and the second reception data is a subset of the first data, the first reception data and the second reception data are not necessarily the same. This is because the first reception data depends on the state of communications between the data transmitting apparatus 100 and the data communication apparatus 200a, and the second reception data depends on the state of communications between the data transmitting apparatus 100 and the data relay apparatus 200b.

The data generating unit 203a generates output data based on at least one of the first and second reception data received. For example, the data generating unit 203a generates the output data by removal of one of the duplicated items of data included in the first and second reception data. Even if the first reception data is a subset of the first data transmitted from the data transmitting apparatus 100, and the second reception data is a subset of the first data transmitted from the data transmitting apparatus 100, the loss of such data included in at least one of the first and second reception data can be prevented.

When, for example, the first user who owns the data communication apparatus 200a is constantly distant from the data transmitting apparatus 100, and the first user who owns the data communication apparatus 200a has few opportunities to remain stationary within the communication area of the data transmitting apparatus 100, the first reception data received by the data communication apparatus 200a tends to be a subset of the first data transmitted from the data transmitting apparatus 100. In addition, when the second user who owns the data relay apparatus 200b frequently stays in the proximity of the data transmitting apparatus 100, or the second user who owns the data relay apparatus 200b remains stationary within the communication area of the data transmitting apparatus 100, the second reception data received by the data relay apparatus 200b tends to be the entirety of the first data transmitted from the data transmitting apparatus 100. In such a case, it may be possible to generate output data corresponding to the first data transmitted from the data transmitting apparatus 100 based only on the second reception data. Alternatively, there is a case where output data corresponding to the first data transmitted from the data transmitting apparatus 100 can be generated, by the removal of one of the duplicated items of data included in the first and second reception data.

### § 2 CONFIGURATION EXAMPLE

<Data Communication System>

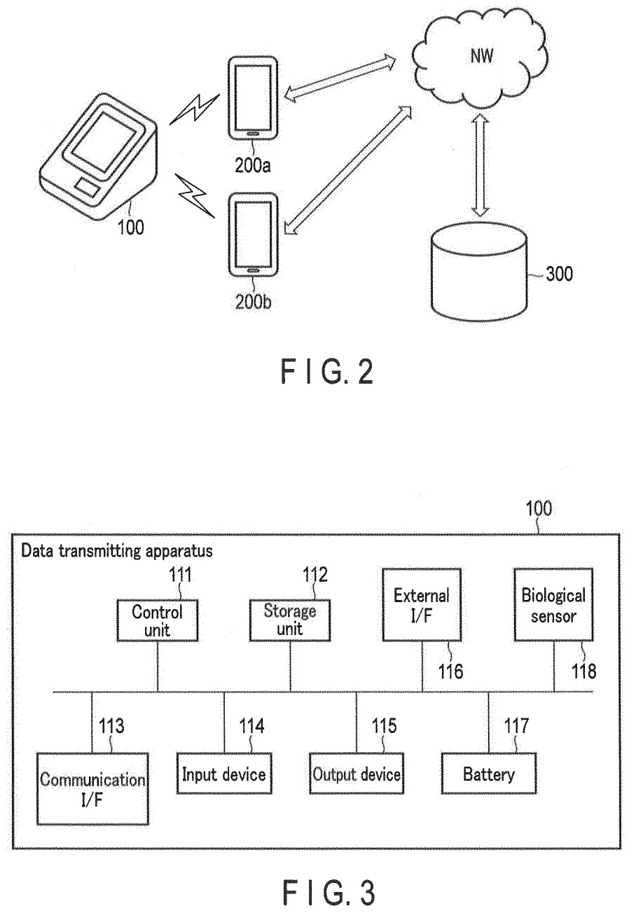

An example of the data communication system according to the present embodiment will be described, with reference to FIG. 2. FIG. 2 is a conceptual diagram illustrating a data communication system including the data transmitting apparatus 100, the data communication apparatus 200a, the data relay apparatus 200b, and the data server 300 according to the present embodiment.

The data transmitting apparatus 100 is a sensor device that routinely measures an amount related to biological information or activity information of the user, such as a blood pressure monitor, a thermometer, an activity tracker, a pedometer, a body composition scale, and a weight scale. The data transmitting apparatus 100 is a device that allows for unidirectional communications such as BLE. In the example of FIG. 2, the appearance of a stationary blood pressure monitor is shown as the data transmitting apparatus 100; however, the data transmitting apparatus 100 is not limited thereto, and may be a wristwatch-type wearable blood pressure monitor, or other sensor device that measures the amount related to biological information or activity information. The data transmitting apparatus 100 transmits, through unidirectional communications, measurement data indicating an amount related to biological information or activity information. It is to be noted that the measurement data corresponds to the transmission data (first data).

The data communication apparatus 200a is a portable information terminal such as a smartphone or a tablet. The data communication apparatus 200a is an apparatus used mainly to enable wireless communications, such as BLE, mobile communications (3G, 4G, etc.), and WLAN.

The data communication apparatus 200a receives, as the first reception data, the first data transmitted from the data transmitting apparatus 100 through a unidirectional communication such as BLE. As described above, the first reception data is either a subset or the entirety of the first data. The data communication apparatus 200a receives second reception data transmitted from the data server 300 through a bidirectional communication, via a network through the employment of mobile communications or WLAN. The data communication apparatus 200a transmits first reception data to the data server 300 through a bidirectional communication, via a network through the employment of mobile communications or WLAN.

The data relay apparatus 200b is a portable information terminal such as a smartphone or a tablet. The data relay apparatus 200b is an apparatus used mainly to enable wireless communications, such as BLE, mobile communications (3G, 4G, etc.), and WLAN.

The data relay apparatus 200b receives, as the second reception data, the first data transmitted from the data transmitting apparatus 100 through a unidirectional communication such as BLE. As described above, the second reception data is either a subset or the entirety of the first data. The data relay apparatus 200b receives the first reception data transmitted from the data server 300 through bidirectional communications, via a network through the employment of mobile communications or WLAN. The data relay apparatus 200b transmits the second reception data to the data server 300 through a bidirectional communication, via a network through the employment of mobile communications or WLAN.

The data server 300 may be a database that manages, based on the first and second reception data, biological information or activity information of a large number of users.

<Data Transmitting Apparatus>

[Hardware Configuration]

Next, an example of a hardware configuration of the data transmitting apparatus 100 according to the present embodiment will be described, with reference to FIG. 3. FIG. 3 schematically shows an example of a hardware configuration of the data transmitting apparatus 100 according to the present embodiment.

As shown in FIG. 3, the data transmitting apparatus 100 is a computer in which a control unit 111, a storage unit 112, a communication interface 113, an input device 114, an output device 115, an external interface 116, a battery 117, and a biological sensor 118 are electrically connected. In FIG. 3, the communication interface and the external interface are respectively denoted as “communication I/F” and “external I/F”.

The control unit 111 includes a central processing unit (CPU), a random access memory (RAM), a read-only memory (ROM), etc. The CPU is an example of a processor. The CPU expands a program stored in the storage unit 112 into the RAM. When the CPU interprets and executes this program, the control unit 111 can execute various information processing operations, such as the processes of functional blocks to be described in item “Software Configuration”.

The storage unit 112 is a so-called “auxiliary storage device”, and may be, for example, a semiconductor memory such as a built-in or external flash memory, a hard disk drive (HDD), or a solid-state drive (SSD). The storage unit 112 stores programs to be executed by the control unit 111, data to be used by the control unit 111, etc. Programs can also be referred to as instructions for operating the control unit 111.

The communication interface 113 includes at least a wireless module that transmits (advertises) a packet through a unidirectional communication such as BLE. The BLE advertising will be described later. The wireless module receives, from the control unit 111, an advertisement packet in BLE in which the transmission data is stored. The wireless module transmits an advertisement packet. The wireless module is also referred to as a “transmitting unit”. It is to be noted that, in the future, BLE may be replaced by other communication standards that allow for low power consumption and unidirectional communications. In that case, the following description may be suitably varied.

The input device 114 is a device for accepting user inputs made via a touch screen, buttons, switches, etc.

The output device 115 is, for example, a device for making outputs from a display, a speaker, etc.

The external interface 116 is a Universal Serial Bus (USB) port, a memory card slot, etc., and is an interface for connection to an external device.

The battery 117 supplies a power-supply voltage for the data transmitting apparatus 100. The battery 117 may be replaceable. It is to be noted that the data transmitting apparatus 100 may be connectable to a commercial power supply via an alternating-current (AC) adapter. In this case, the battery 117 can be omitted.

The biological sensor 118 obtains measurement data by measuring an amount related to the user's biological information. The operation of the biological sensor 118 is controlled by, for example, an unillustrated sensor controller. The measurement data is stored in the storage unit 112 in association with the date-and-time data. The biological sensor 118 typically includes a blood pressure sensor that obtains blood pressure data by measuring an amount related to the user's blood pressure. In this case, the measurement data includes blood pressure data. The blood pressure data may include, for example, values of the systolic blood pressure (SBP) and the diastolic blood pressure (DBP), as well as the pulse rate, but is not limited thereto. In addition, the measurement data can include electrocardiogram data, pulse wave data, body temperature data, etc.

The blood pressure sensor can include a blood pressure sensor (hereinafter also referred to as a “continuous blood pressure sensor”) capable of continuously measuring an amount related to the user's blood pressure per beat. The continuous blood pressure sensor may continuously measure an amount related to the user's blood pressure from a pulse transit time (PTT), or may implement continuous measurement through the tonometry technique or other techniques.

The blood pressure sensor may include a blood pressure sensor that cannot perform continuous measurements (hereinafter also referred to as a “discontinuous blood pressure sensor”), either in place of or in addition to the continuous blood pressure sensor. A discontinuous blood pressure sensor measures an amount related to a user's blood pressure using, for example, a cuff as a pressure sensor (oscillometric method).

A discontinuous blood pressure sensor (in particular, an oscillometric blood pressure sensor) tends to provide higher measurement accuracy than a continuous blood pressure sensor. Thus, the blood pressure sensor may be configured to measure the blood pressure data with high precision, triggered by satisfaction of a certain condition (e.g., when the user's blood pressure data obtained by measurement of the continuous blood pressure sensor indicates a predetermined state), by operating a discontinuous blood pressure sensor in place of a continuous blood pressure sensor.

It should be noted that, regarding the specific hardware configuration of the data transmitting apparatus 100, the components can be suitably omitted, replaced, or added, according to the embodiment. The control unit 111 may include, for example, a plurality of processors. The data transmitting apparatus 100 may be configured of a plurality of sensor devices.

[Software Configuration]

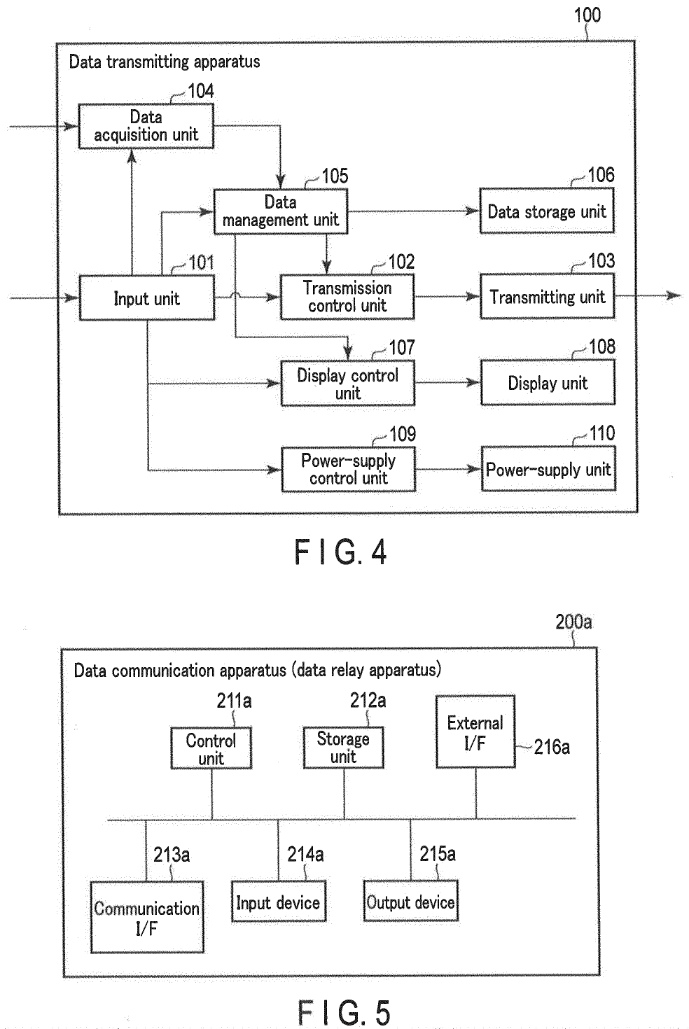

Next, an example of a software configuration of the data transmitting apparatus 100 according to the present embodiment will be described, with reference to FIG. 4. FIG. 4 schematically shows an example of a software configuration of the data transmitting apparatus 100.

The control unit 111 in FIG. 3 expands the program stored in the storage unit 112 into the RAM. Thereafter, the control unit 111 causes the CPU to interpret and execute the program, and controls various hardware elements shown in FIG. 3. Thereby, as shown in FIG. 4, the data transmitting apparatus 100 functions as a computer including an input unit 101, a transmission control unit 102, a transmitting unit 103, a data acquisition unit 104, a data management unit 105, a data storage unit 106, a display control unit 107, a display unit 108, a power-supply control unit 109, and a power-supply unit 110.

The data acquisition unit 104 acquires biological data output from the biological sensor 118, and outputs the acquired biological data to the data management unit 105.

The data management unit 105 receives the biological data, and writes the received biological data into the data storage unit 106. Also, the data management unit 105 generates, based on a user input, a packet containing transmission data, and inputs the packet to the transmission control unit 102. The transmission data includes biological data and date-and-time data associated with the biological data. A configuration may be adopted in which the transmission control unit 102 generates a packet in advance regardless of a user input, the data storage unit 106 stores data in the packet, and the transmission control unit 102 reads, based on the user input, the packet from the data storage unit 106 to allow the packet to be input to the transmission control unit 102.

The data management unit 105 may read the biological data stored in the data storage unit 106, triggered by an instruction from the transmission control unit 102 or the display control unit 107, and then transmit the biological data to the transmission control unit 102 or the display control unit 107.

The data storage unit 106 stores biological data written by the data management unit 105. The data storage unit 106 stores the packet written by the data management unit 105. When biological data is newly stored, the data management unit 105 may automatically send the biological data to the display control unit 107.

The input unit 101 accepts user inputs. For example, the input unit 101 accepts a first user input that instructs transmission of first data, and sends the first user input to the transmission control unit 102, etc. The input unit 101 accepts a second user input that instructs termination of the operation, and sends the second user input to the transmission control unit 102, etc. The input unit 101 accepts a third user input that controls data display on the display unit 108 and a fourth user input that instructs commencement of measurement by the biological sensor 118.

Based on the first user input, the transmission control unit 102 instructs execution of transmission of the packet, and inputs the generated packet or the packet read from the data storage unit 106 to the transmitting unit 103. For example, the transmission control unit 102 instructs, based on the first user input, repeated transmission of the packet over a period of time until the second user input is accepted.

In the case of instructing execution of transmission of the packet, the transmission control unit 102 notifies the data management unit 105 of the unique identification information of the packet, and the data management unit 105, based on the notification, manages the packet as one which has already been transmitted.

The transmitting unit 103 transmits, through a unidirectional communication compliant with a communication standard such as BLE, a radio signal that carries a packet defined by the standard. Based on an instruction to repeatedly transmit a packet, for example, the transmitting unit 103 repeatedly transmits (advertises) a packet for unidirectional communications.

The display control unit 107 generates display data based on the user input from the input unit 101 and the data from the data management unit 105, and inputs the generated display data to the display unit 108. The display unit 108 displays an image based on display data input from the display control unit 107. For example, the display control unit 107 reads, based on the third user input, biological data from the data storage unit 106 and generates display data for the display unit 108 based on the read biological data. The display unit 108 then displays an image corresponding to the biological data based on the generated display data.

The power-supply control unit 109 commences, based on the user input that instructs commencement of supply of the power-supply voltage from the input unit 101, the supply of the power-supply voltage, and instructs, based on the user input that instructs termination of the supply of the power-supply voltage from the input unit 101, the termination of the supply of the power-supply voltage.

The power-supply unit 110 commences the supply of the power-supply voltage based on the instruction to commence supply of the power-supply voltage from the power-supply control unit 109, and terminates the supply of the power-supply voltage based on the instruction to terminate the supply of the power-supply voltage from the power-supply control unit 109.

<Data Communication Apparatus>

[Hardware Configuration]

Next, an example of a hardware configuration of the data communication apparatus 200a according to the present embodiment will be described, with reference to FIG. 5. FIG. 5 schematically shows an example of a hardware configuration of the data communication apparatus 200a. The data communication apparatus 200a and the data relay apparatus 200b may have the same configuration; in the present embodiment, a case will be described where the data communication apparatus 200a and the data relay apparatus 200b have the same configuration, and the description of the hardware configuration of the data relay apparatus 200b will be omitted.

As shown in FIG. 5, the data communication apparatus 200a is a computer in which a control unit 211a, a storage unit 212a, a communication interface 213a, an input device 214a, an output device 215a, and an external interface 216a are electrically connected. In FIG. 5, the communication interface and the external interface are respectively denoted as “communication I/F” and “external I/F”.

The control unit 211a includes a CPU, a RAM, a ROM, etc. The CPU is an example of a processor. The CPU expands the program stored in the storage unit 212a into the RAM. When the CPU interprets and executes this program, the control unit 211a can execute various information processing operations, such as the processes of functional blocks to be described in item “Software Configuration”.

The storage unit 212a is a so-called auxiliary storage device, and may be, for example, a semiconductor memory such as a built-in or external flash memory. The storage unit 212a stores programs to be executed by the control unit 211a, data to be used by the control unit 211a, etc. Programs can also be referred to as instructions for operating the control unit 211a.

The communication interface 213a includes various wireless communication modules, which are mainly for BLE, mobile communications (e.g., 3G and 4G), WLAN, etc. The communication interface 213a may further include a wired communication module such as a wired local area network (LAN) module. The communication module for BLE receives, from the data transmitting apparatus 100, first reception data (e.g., an advertisement packet) through a unidirectional communication. The communication module for BLE may also be referred to as a “receiving unit”.

A communication module for mobile communications, WLAN, etc. receives second reception data including measurement data from the data server 300 through a bidirectional communication. When a communication module for mobile communications, WLAN, etc. receives second reception data from the data server 300 through a bidirectional communication, the communication module may be referred to as a “transmitting and receiving unit”. A communication module for mobile communications, WLAN, etc. transmits first reception data to the data server 300 through bidirectional communication. When a communication module for mobile communications, WLAN, etc. transmits the first reception data to the data server 300 through a bidirectional communication, the communication module may be also referred to as a “relay unit”.

The input device 214a is a device for accepting a user input such as a touch screen.

The output device 215a is, for example, a device for making outputs from a display, a speaker, etc.

The external interface 216a is a USB port, a memory card slot, etc., and is an interface for connection to an external device.

It should be noted that, regarding the specific hardware configuration of the data communication apparatus 200a, the components can be suitably omitted, replaced, or added, according to the embodiment. For example, the control unit 211a may include a plurality of processors. The data communication apparatus 200a may be configured of a plurality of information processing devices. As the data communication apparatus 200a, a general-purpose tablet personal computer (PC), etc., as well as an information processing device designed exclusively for the services to be provided, may be used.

[Software Configuration]

Next, an example of a software configuration of the data communication apparatus 200a according to the present embodiment will be described, with reference to FIG. 6. FIG. 6 schematically shows an example of a software configuration of the data communication apparatus 200a. The data communication apparatus 200a and the data relay apparatus 200b may have the same configuration; in the present embodiment, a case will be described where the data communication apparatus 200a and the data relay apparatus 200b have the same configuration, and the description of the software configuration of the data relay apparatus 200b will be omitted.

The control unit 211a in FIG. 5 expands the program stored in the storage unit 212a into the RAM. Thereafter, the control unit 211a causes the CPU to interpret and execute the program, and controls various hardware elements shown in FIG. 5. Thereby, as shown in FIG. 6, the data communication apparatus 200a functions as a computer including a receiving unit 201a, a transmitting and receiving unit 202a, a data generating unit 203a, a data processing unit 204a, a relay control unit 205a, a data storage unit 206a, an input unit 207a, a display control unit 208a, and a display unit 209a.

The receiving unit 201a receives a radio signal that carries a packet from the data transmitting apparatus 100 through a unidirectional communication. This packet is, for example, an advertisement packet in BLE. It is to be noted that, in the future, BLE may be replaced by other communication standards that allow for low power consumption and unidirectional communications. In that case, the following description may be suitably varied.

A schematic description of BLE advertisement will be given below.

In the passive scanning mode adopted in BLE, a new node periodically transmits advertisement packets to indicate its presence, as illustrated in FIG. 9. By entering a sleep state, which consumes low power, the new node can conserve power consumption during the period from transmission of an advertisement packet to transmission of a subsequent advertisement packet. Since the receiver side of advertisement packets operate intermittently, the power consumption incurred in transmission and reception of advertisement packets is low.

FIG. 10 shows a basic configuration of a BLE wireless communication packet. A BLE wireless communication packet contains a 1-byte preamble, a 4-byte Access Address, a 2-to-39-byte (variable) Protocol Data Unit (PDU), and a 3-byte Cyclic Redundancy Checksum (CRC). The length of the BLE wireless communication packet depends on the length of the PDU, and ranges from 10 to 47 bytes. A 10-byte BLE wireless communication packet (with a 2-byte PDU) is also called an “Empty PDU packet”, and is periodically exchanged between the master and the slave.

The preamble field is prepared for synchronization in BLE wireless communications, and stores repetitions of “01” or “10”. For the Access Address, fixed numerical values are stored in an advertising channel, and random numbers are stored in a data channel. In the present embodiment, an advertisement packet that is a BLE wireless communication packet to be transmitted on the advertising channel is targeted. The CRC field is used for detection of reception errors. The range of calculation by the CRC is only the PDU field.

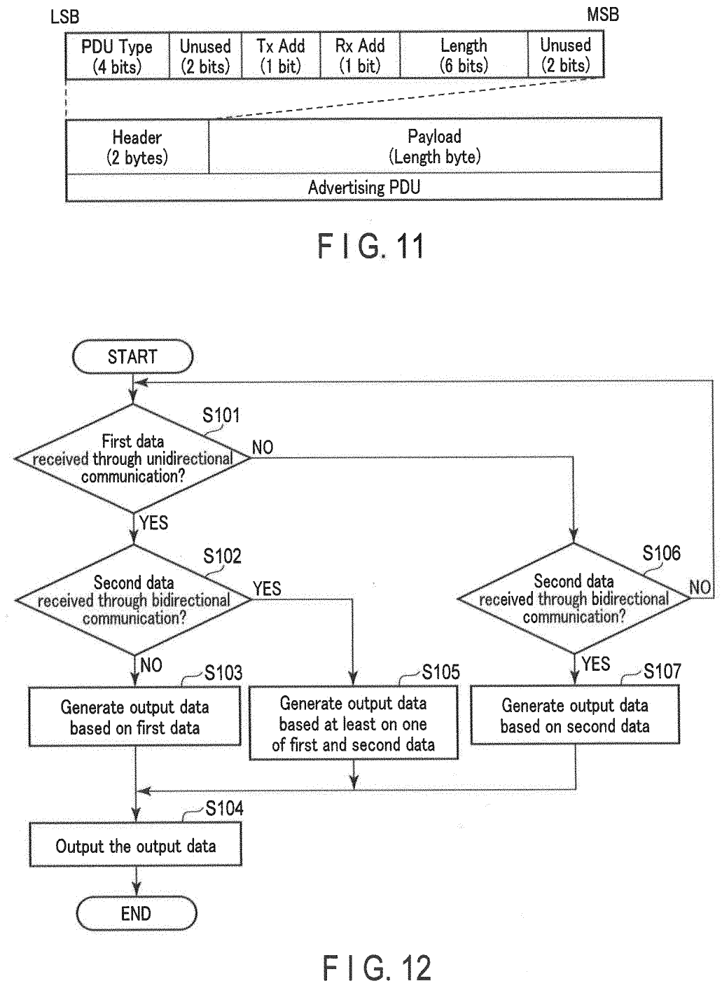

Next, the PDU field of an advertisement packet will be described, with reference to FIG. 11.

The PDU field of an advertisement packet contains a 2-byte header and a 0-to-37-byte (variable) payload. The header further includes a 4-bit PDU Type field, a 2-bit unused field, a 1-bit TxAdd field, a 1-bit RxAdd field, a 6-bit Length field, and a 2-bit unused field.

A value indicating the type of the PDU is stored in the PDU Type field. Some values such as “connectable advertising” and “non-connectable advertising” have been defined. A flag indicating whether or not a transmission address is present in the payload is stored in the TxAdd field. Similarly, a flag indicating whether or not a reception address is present in the payload is stored in the RxAdd field. In the Length field, a value indicating the byte size of the payload is stored.

The payload can store given data. Accordingly, the data transmitting apparatus 100 stores the biological data and the date-and-time data in the payload using a predetermined data structure. The data structure may include, for example, an identifier indicating the user, an identifier indicating the data transmitting apparatus 100 from which the data is transmitted, an identifier indicating the data communication apparatus 200a (or the data relay apparatus 200b) for which the data is destined, the date-and-time data, and one or more types of measurement data such as the systolic blood pressure and the diastolic blood pressure associated with the date-and-time data, the pulse rate, and the amount of activity.

Returning to the description of the software configuration of the data communication apparatus 200a, the receiving unit 201a performs reception processing including low-noise amplification, filtering, down-conversion, etc. on a radio signal, and obtains a reception signal in the intermediate frequency bandwidth or the baseband bandwidth. The receiving unit 201a sends the first reception data included in the reception signal to the data generating unit 203a.

The receiving unit 201a reproduces a BLE advertisement packet transmitted from the data transmitting apparatus 100 by performing demodulation and decoding on the reception signal. Thereafter, the receiving unit 201a extracts the PDU payload from the BLE advertisement packet.

By checking, for example, the identifier (indicating the apparatus from which the measurement data is transmitted or the valid destination) contained in the payload, the receiving unit 201a may discard the received packet if the value of the identifier is inappropriate. If the value of the identifier is appropriate, the receiving unit 201a inputs the data extracted from the BLE advertisement packet to the data generating unit 203a. The receiving unit 201a inputs the extracted data to the data generating unit 203a based on, for example, advance settings (data generation settings).

The transmitting and receiving unit 202a receives the second reception data from the data server 300 through bidirectional communications, and inputs the second reception data to the data generating unit 203a based on advance settings (data generation settings).

The data generating unit 203a generates output data based on at least one of the first and second reception data. For example, the data generating unit 203a generates the output data by removal of one of the duplicated items of data included in the first and second reception data. Thereby, the loss of the first data transmitted from the data transmitting apparatus 100 can be compensated for. The data generating unit 203a sends the output data to the data processing unit 204a.

The processing of removing one of the duplicated items of data included in the first and second reception data will be described. The packet transmitted from the data transmitting apparatus 100 contains, for example, biological data and date-and-time data associated with the biological data. The data generating unit 203a determines, from the items of biological data included in the first and second reception data, that the items of biological data associated with the same date-and-time data are duplicated items of data, and generates output data by removing one of the duplicated items of data. When the packet transmitted from the data transmitting apparatus 100 contains biological data and identification data associated with the biological data, the data generating unit 203a determines, from the items of biological data included in the first and second reception data, that the items of biological data associated with the identical identification data are duplicated items of data, and generates output data by removal of one of the duplicated items of data.

The data processing unit 204a sends the output data to the data storage unit 206a. The data storage unit 206a stores the output data. The date-and-time data and the biological data, for example, included in the output data are stored in association with each other. When a read request is received, the stored output data is output.

In accordance with an instruction from, for example, an unillustrated high-order application (e.g., an application that manages biological data), the data processing unit 204a reads output data stored in the data storage unit 206a, and inputs the read output data to the display control unit 208a.

The display control unit 208a generates display data based on the output data, and inputs the generated display data to the display unit 209a. The display unit 209a displays an image based on display data input from the display control unit 208a.

The data generating unit 203a does not execute data generation, based on in-advance settings (in which data generation is unset and data relay is set). The data processing unit 204a inputs the first reception data to the relay control unit 205a, based on in-advance settings (in which data generation is unset and data relay is set). The relay control unit 205a performs control to transmit the first reception data to the data relay apparatus 200b, which is a preset destination apparatus, via the data server 300 through bidirectional communications. The transmitting and receiving unit 202a transmits the first reception data to the data server 300 through bidirectional communications, according to the control of the relay control unit 205a.

<Data Server>

[Hardware Configuration]

Next, an example of a hardware configuration of the data server 300 according to the present embodiment will be described, with reference to FIG. 7. FIG. 7 schematically shows an example of a hardware configuration of the data server 300 according to the present embodiment.

As shown in FIG. 7, the data server 300 is a computer in which a control unit 311, a storage unit 312, a communication interface 313, an input device 314, an output device 315, and an external interface 316 are electrically connected. In FIG. 7, the communication interface and the external interface are respectively denoted as “communication I/F” and “external I/F”.

The control unit 311 includes a central processing unit (CPU), a random access memory (RAM), a read-only memory (ROM), etc. The CPU is an example of a processor. The CPU expands a program stored in the storage unit 312 into the RAM. When the CPU interprets and executes this program, the control unit 311 can execute various information processing operations, such as the processes of functional blocks to be described in item “Software Configuration”.

The storage unit 312 is a so-called auxiliary storage device, and may be, for example, a semiconductor memory such as a built-in or external flash memory, a hard disk drive (HDD), or a solid-state drive (SSD). The storage unit 312 stores programs to be executed by the control unit 311, data to be used by the control unit 311, etc. Programs can also be referred to as instructions for operating the control unit 311.

The communication interface 313 includes various wireless communication modules, which are mainly for mobile communications (e.g., 3G and 4G), WLAN, etc. The communication interface 313 may further include a wired communication module such as a wired local area network (LAN) module. A communication module for mobile communications, WLAN, etc. receives second reception data transmitted from the data relay apparatus 200b through a bidirectional communication, and transmits second reception data to the data communication apparatus 200a through bidirectional communications. In this case, the communication module may also be referred to as a “transmitting and receiving unit”. Alternatively, a communication module for mobile communications, WLAN, etc. receives first reception data transmitted from the data communication apparatus 200a through a bidirectional communication, and transmits first reception data to the data relay apparatus 200b through bidirectional communications. In this case, the communication module is sometimes referred to as a “relay unit”.

The input device 314 is a device for receiving user inputs such as a touch screen, buttons, and switches.

The output device 315 is, for example, a device for making outputs from a display, a speaker, etc.

The external interface 316 is a Universal Serial Bus (USB) port, a memory card slot, etc., and is an interface for connection to an external device.

It should be noted that, regarding the specific hardware configuration of the data server 300, the components can be suitably omitted, replaced, or added, according to the embodiment. For example, the control unit 311 may include a plurality of processors.

[Software Configuration]

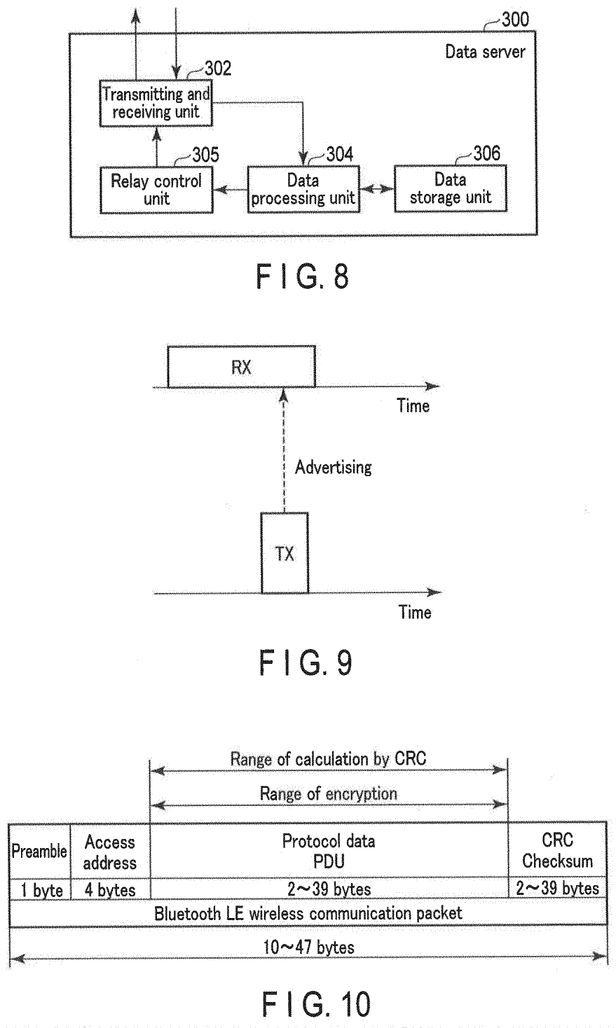

Next, an example of the software configuration of the data server 300 according to the present embodiment will be described with reference to FIG. 8. FIG. 8 schematically shows an example of the software configuration of the data server 300.

The control unit 311 in FIG. 7 expands the program stored in the storage unit 312 into the RAM. Thereafter, the control unit 311 causes the CPU to interpret and execute the program, and controls various hardware elements shown in FIG. 7. Thereby, as shown in FIG. 8, the data server 300 functions as a computer including a transmitting and receiving unit 302, a data processing unit 304, a relay control unit 305, and a data storage unit 306.

The transmitting and receiving unit 302 receives second reception data transmitted from the data relay apparatus 200b through bidirectional communications, and outputs the second reception data to the data processing unit 304. The data processing unit 304 inputs the second reception data to the data storage unit 306, and the data storage unit 306 stores the second reception data. The data processing unit 304 inputs the second reception data to the relay control unit 305, and the relay control unit 305 performs control to transmit the second reception data to the data communication apparatus 200a based on advance settings (settings for relay from the data relay apparatus 200b to the data communication apparatus 200a). The transmitting and receiving unit 302 transmits the second reception data to the data communication apparatus 200a through bidirectional communications.

The transmitting and receiving unit 302 receives the first reception data transmitted from the data communication apparatus 200a through bidirectional communications, and outputs the first reception data to the data processing unit 304. The data processing unit 304 inputs the first reception data to the data storage unit 306, and the data storage unit 306 stores the first reception data. The data processing unit 304 inputs the first reception data to the relay control unit 305, and the relay control unit 305 performs control to transmit the first reception data to the data relay apparatus 200b based on advance settings (settings for relay from the data communication apparatus 200a to the data relay apparatus 200b). The transmitting and receiving unit 302 transmits the first reception data to the data relay apparatus 200b through bidirectional communications.

<Others>

In the present embodiment, an example has been described where the functions of the data transmitting apparatus 100, the data communication apparatus 200a, the data relay apparatus 200b, and the data server 300 is realized by a general-purpose CPU. However, some or all of the above functions may be realized by one or a plurality of dedicated processors. Regarding the software configuration of each of the data transmitting apparatus 100, the data communication apparatus 200a, the data relay apparatus 200b, and the data server 300, functions may be suitably omitted, replaced, or added according to the embodiment.

### § 3 EXAMPLE OF OPERATION

<Data Communication Apparatus>

Next, an example of the data receiving operation of the data communication apparatus 200a will be described, with reference to FIG. 12. FIG. 12 is a flowchart showing an example of a data receiving operation of the data communication apparatus 200a. It is to be noted that the processing procedure to be described below is merely an example, and each process may be varied where possible. In the processing procedure to be described below, the steps may be suitably omitted, substituted, and/or added, according to the embodiment.

As shown in FIG. 12, the receiving unit 201a of the data communication apparatus 200a receives the first data transmitted from the data transmitting apparatus 100 through a unidirectional communication (step S101, YES), and if the transmitting and receiving unit 202a does not receive the second data transmitted from the data server 300 through a bidirectional communication (step S102, NO), the data generating unit 203a generates output data based on the first data (step S103), and the data processing unit 204a outputs the output data (step S104). The data storage unit 206a stores, for example, the output data. The display control unit 208a generates, based on the output data, display data, and the display unit 209a then displays the display data. The data communication apparatus 200a receives a subset or the entirety of the first data, and the subset or the entirety of the first data received by the data communication apparatus 200a is the first reception data, as described above. That is, the data generating unit 203a generates output data based on the first reception data.

The receiving unit 201a of the data communication apparatus 200a receives the first data transmitted from the data transmitting apparatus 100 through a unidirectional communication (step S101, YES), and if the transmitting and receiving unit 202a receives the second data transmitted from the data server 300 through a bidirectional communication (step S102, YES), the data generating unit 203a generates output data based on at least one of the first and second data (step S105), and the data processing unit 204a outputs the output data (step S104). The subset or entirety of the first data received by the data communication apparatus 200a is the above-described second reception data. That is, the data generating unit 203a generates output data based on at least one of the first and second reception data.

If the receiving unit 201a of the data communication apparatus 200a does not receive the first data transmitted from the data transmitting apparatus 100 through a unidirectional communication (step S101, NO) and the transmitting and receiving unit 202a receives the second data transmitted from the data server 300 through a bidirectional communication (step S106, YES), the data generating unit 203a generates output data based on the second data (step S107), and the data processing unit 204a outputs the output data (step S104). That is, the data generating unit 203a generates output data based on the second reception data.

<Data Relay Apparatus>

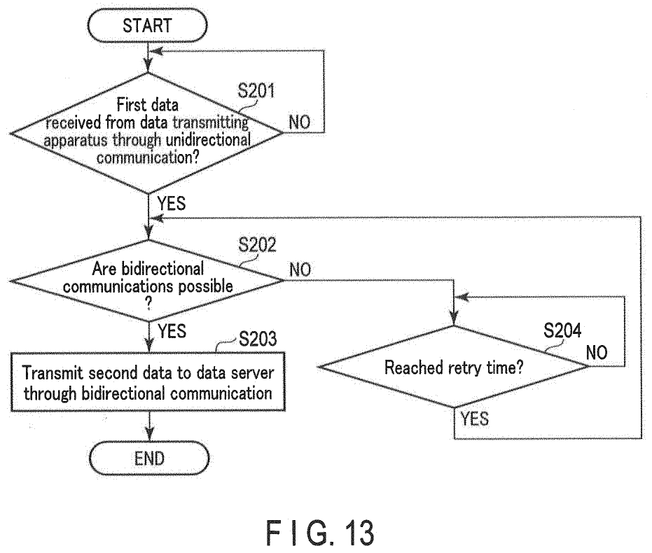

Next, an example of a relay operation of the data relay apparatus 200b will be described, with reference to FIG. 13. FIG. 13 is a flowchart illustrating an example of a relay operation of the data relay apparatus 200b. It is to be noted that the processing procedure to be described below is merely an example, and each process may be varied where possible. In the processing procedure to be described below, the steps may be suitably omitted, substituted, and/or added, according to the embodiment.

As shown in FIG. 13, the receiving unit 201b of the data relay apparatus 200b receives first data transmitted from the data transmitting apparatus 100 through a unidirectional communication (step S201, YES). At this time, the data relay apparatus 200b receives a subset or the entirety of the first data, and the subset or the entirety of the first data received by the data relay apparatus 200b is the above-described second reception data. If bidirectional communications with the data server 300 are possible (step S202, YES), the transmitting and receiving unit 202b transmits the second data (second reception data) to the data server 300 through bidirectional communications (step S203).

If bidirectional communications with the data server 300 are not possible (step S202, NO) and the retry time has been reached (step S204, YES), it is determined whether bidirectional communications with the data server 300 are possible; if so, (step S202, YES), the transmitting and receiving unit 202b transmits the second data (second reception data) to the data server 300 through bidirectional communications (step S203).

[Functions and Effects]

As described above, according to the present embodiment, since the data communication apparatus is provided with an opportunity to receive data from the data transmitting apparatus either directly or indirectly through the employment of both unidirectional and bidirectional communications, it is possible to increase the opportunity to receive data from the data transmitting apparatus. Since the output data is generated based on at least one of the first reception data received through a unidirectional communication with the data transmitting apparatus and the second reception data received through a bidirectional communication with the data server, it is possible to compensate for some or all of the data reception failures (or increase the possibility of the compensation) when reception of at least one of the first data and the second data has failed. When at least one of the first and second reception data cannot be received due to the communication status, the output data can be generated based on the other data that has been received. It is thus possible to provide high-value output data with little or no leakage.

By allowing the person targeted for measurement of the blood pressure data to own the data communication apparatus and a family member of the targeted person to own the data relay apparatus, thereby providing a plurality of opportunities (multiple systems) to receive data from the data transmitting apparatus, it is possible, when reception of data has failed, to compensate for some or all of the data reception failures (or increase the possibility of the compensation).

### § 4 MODIFICATION

The embodiment of the present invention has been described in detail above; however, in every respect, the description given above is merely an illustration of the present invention. As a matter of course, various alternations and modifications can be made, without departing from the spirit of the invention. That is, in implementing the present invention, a specific configuration may be suitably adopted according to the above embodiment. The data appearing in the above embodiment has been described with natural language; however, in actuality, it is represented by pseudo language, a command, a parameter, machine language, etc. that can be recognized by a computer.

For example, in the present embodiment, a case has been described where the second reception data is transmitted to the data communication apparatus via the data server from a single data relay device; however, a plurality of items of second reception data may be transmitted from a plurality of data relay apparatuses to the data communication apparatus via the data server. By using a plurality of data relay devices, it is possible to increase opportunities to receive data from the data transmitting apparatus. It is thereby possible, when reception of data has failed, to compensate for some or all of the data reception failures (or increase the possibility of the compensation).

The data relay device may be a gateway device. This gateway device is a device that supports BLE and interconnects networks through the use of different network protocol technologies. The gateway device receives data from the data transmitting apparatus through unidirectional communications, and accumulates the received data. Let us assume that the data accumulated in the gateway device is the third reception data. The gateway device transmits the third reception data to the data server through a bidirectional communication.

The data communication apparatus receives the third reception data accumulated in the gateway device through a bidirectional communication. The data communication apparatus may receive third reception data accumulated in the data server through a bidirectional communication. The data communication apparatus generates output data based on at least one of the above-described first reception data and third reception data. It is thereby possible to increase the opportunity to receive data from the data transmitting apparatus. It is thus possible, when reception of data has failed, to compensate for some or all of the data reception failures (or increase the possibility of the compensation).

In the present embodiment, a case has been described where biological data, etc. is transmitted from the data transmitting apparatus through unidirectional communications; however, the data to be transmitted from the data transmitting apparatus through unidirectional communications is not limited to biological data such as blood pressure data. For example, support information regarding the measurement may be transmitted from the data transmitting apparatus through a unidirectional communication. The data communication apparatus may be configured to receive and display the support information. The support information includes, for example, information on at least one of the last date and time of measurement and the period of time that has passed since the last date and time of measurement. The support information may include individual identification information (e.g., name). It is thereby possible to prompt the user of the data communication apparatus to measure the amount related to the blood pressure, etc. Since support information can be received by any data communication apparatus compatible with unidirectional communications, if a data communication apparatus of a user other than a user who has been neglecting to measure the amount related to the blood pressure (hereinafter also referred to as a “blood pressure measurement”) has received the support information, the user other than the user who has been neglecting to measure the blood pressure may prompt the user who has been neglecting the blood pressure measurement to perform blood pressure measurement.

### § 5 ADDITIONAL DESCRIPTIONS

Some or all of the above embodiments may be described as in the additional descriptions to be given below, as well as the claims; however, the embodiments are not limited thereto.

(Additional Description 1)

A data communication system comprising:

first and second data communication apparatuses; and

a data server which communicates with the first and second data communication apparatuses, wherein

each of the first and second data communication apparatuses and the data server includes:

a memory; and

a processor connected to the memory, and

the processor of the first data communication apparatus is configured to function as an apparatus that receives first data transmitted from the data transmitting apparatus through a unidirectional communication, receives second data transmitted from the data server through a bidirectional communication, and generates output data based on at least one of the first and second data,

the processor of the second data communication apparatus is configured to function as an apparatus that receives the second data transmitted from the data transmitting apparatus through a unidirectional communication, and transmits the second data to the data server through a bidirectional communication, and

the processor of the data server is configured to function as a server that receives the second data transmitted from the second data communication apparatus through a bidirectional communication, and transmits the second data to the first data communication apparatus through a bidirectional communication.

### REFERENCE SIGNS LIST

100: Data transmitting apparatus
101: Input unit
102: Transmission control unit
103: Transmitting unit
104: Data acquisition unit
105: Data management unit
106: Data storage unit
107: Display control unit
108: Display unit
109: Power-supply control unit
110: Power-supply unit
111: Control unit
112: Storage unit
113: Communication interface
114: Input device
115: Output device
116: External interface
117: Battery
118: Biological sensor
200a: Data communication apparatus
200b: Data relay apparatus
201a: Receiving unit
201b: Receiving unit
202a: Transmitting and receiving unit
202b: Transmitting and receiving unit
203a: Data generating unit
203b: Data generating unit
204: Data processing unit
204a: Data processing unit
204b: Data processing unit
205a: Relay control unit
206a: Data storage unit
207a: Input unit
208a: Display control unit
209a: Display unit
211a: Control unit
212a: Storage unit
213a: Communication interface
214a: Input device
215a: Output device
216a: External interface
300: Data server
302: Transmitting and receiving unit
304: Data processing unit
305: Relay control unit
306: Data storage unit
311: Control unit
312: Storage unit
313: Communication interface
314: Input device
315: Output device
316: External interface

1. A data communication system comprising:
first and second data communication apparatuses; and
a data server which communicates with the first and second data communication apparatuses, wherein
the first data communication apparatus receives first data transmitted from a data transmitting apparatus through a unidirectional communication, receives second data transmitted from the data server through a bidirectional communication, and generates output data based on at least one of the first and second data,
the second data communication apparatus receives the second data transmitted from the data transmitting apparatus through a unidirectional communication, and transmits the second data to the data server through a bidirectional communication, and
the data server receives the second data transmitted from the second data communication apparatus through a bidirectional communication, and transmits the second data to the first data communication apparatus through a bidirectional communication.

2. The data communication system according to claim 1, wherein the first data communication apparatus generates the output data by removal of one of the duplicated items of data included in the first and second data.

3. The data communication system according to claim 1, wherein the first and second data include biological data.

4. The data communication system according to claim 1, wherein the first data communication apparatus receives the second data from the data server through a periodic bidirectional communication.

5. The data communication system according to claim 1, wherein the first data communication apparatus transmits the first data to the data server through the bidirectional communication, based on the reception of the first data.

6. A data communication apparatus comprising:
a receiving circuit which receives first data transmitted from a data transmitting apparatus through a unidirectional communication;
a transmitting and receiving circuit which receives second data transmitted from a data server through a bidirectional communication; and
a data generating circuit which determines, from items of biological data included in the first and second data, that items of biological data associated with identical date-and-time data are duplicated items of data, and generates output data based on the first and second data by removal of one of the duplicated items of data, wherein
the second data is data obtained by another data communication apparatus different from said data communication apparatus through reception of the first data transmitted from the data transmitting apparatus through the unidirectional communication, and is data obtained by said another data communication apparatus and transmitted to the data server.

7. A data communication apparatus comprising:
a receiving circuit which receives first data transmitted from a data transmitting apparatus through a unidirectional communication;
a transmitting and receiving circuit which receives second data transmitted from a data server through a bidirectional communication;
a data generating circuit which determines, from items of biological data included in the first and second data, that items of biological data associated with identical identification data are duplicated items of data, and generates output data based on the first and second data by removal of one of the duplicated items of data, wherein
the second data is data obtained by another data communication apparatus different from said data communication apparatus through reception of the first data transmitted from the data transmitting apparatus through the unidirectional communication, and is the data obtained by said another data communication apparatus and transmitted to the data server.

8. The data communication apparatus according to claim 6, wherein the transmitting and receiving circuit receives the second data from the data server through a periodic bidirectional communication.

9. The data communication apparatus according to claim 6, comprising a relay control circuit which performs control to transmit the first data to the data server through the bidirectional communication, based on the reception of the first data by the receiving circuit.
